(19) United States
(12) Patent Application Publication
(10) Pub. No.: US 2010/0203136 A1
(43) Pub. Date: Aug. 12, 2010

(54) METHOD FOR DELIVERING A HUMAN CHORIONIC GONADOTROPIN (Hcg) VACCINE FOR LONG-ACTING ANTIBODY PROTECTION

(75) Inventor: Garfield P. Royer, Upperville, VA (US)

Correspondence Address: C. Rachal Winger;c/o OCIP Group, 1900 Main Street, Suite 600, Irvine, CA, 92614-7319, US

(73) Assignee: ROYER BIOMEDICAL, INC.

(21) Appl. No.: 12/299,721

(22) PCT Filed: May 2, 2007
(86) PCT No.: PCT/US2007/067994

§ 371 Date: Apr. 30, 2010

Related U.S. Application Data
(60) Provisional application No. 60/797,458, filed on May 4, 2006.

(51) Int. Cl. A61K 9/00; A61K 39/00; A61K 39/385; A61P 35/00

(52) U.S. Cl. 424/484; 424/184.1; 424/195.11

(57) ABSTRACT

This invention comprises a method of immunization in which human chorionic gonadotropin (hCG) vaccine antigens are incorporated into an inorganic salt/biopolymer complex using a simple manufacturing process. The resulting solid matrix is administered to human subjects in the form of microparticles. The method comprises suspending microparticles in an emulsion of a natural oil and water containing an adjuvant compound acceptable for human use and injecting a pharmaceutical dose of the suspension intramuscularly. The vaccine antigens are conjugates of peptide fragments of the beta subunit of hCG with a carrier protein, diphtheria toxoid. Vaccine antigens delivered in this formulation are effective for eliciting antibodies in recipients for the treatment of cancer or hormone-related diseases.

### BACKGROUND OF THE INVENTION

The application of many newly developed vaccines has been hindered by the lack of a suitable adjuvant/delivery system to render them efficacious and safe for medical use. Numerous new vaccines have been developed utilizing fragments or subunits of antigen molecules, prepared by recombinant DNA technology or peptide synthesis, which have been shown to be effective when immune responses to them are adequate. In many situations, the requirements to elicit an adequate immune response (antibodies or cellular activation) involve the use of adjuvants or vehicles that produce unacceptable local inflammatory reactions at the injection site or other undesirable side effects. The only approved adjuvants for use in human vaccines are aluminum salts, which when absorbed to the antigen, enhance the immune response to the antigen. Unfortunately, these adjuvants are sufficiently effective for only a limited number of antigens and are rarely useful for new vaccines employing subunit or peptide antigens. In general, these vaccines are weakly immunogenic and require a more potent adjuvant effect than is provided by aluminum salts. When other commonly available adjuvants are used, such as mineral oil/water emulsions, efficacy can be demonstrated, but side effects, such as pain and tissue inflammation at the injection site, often preclude their general use in humans.

The production of an immune response to a given antigen depends on a series of biological events, including: delivery of the antigen to a site accessible to lymphoid cells; attraction of these cells to the microenvironment where recognition of the antigen as foreign by T lymphocytes (T-cells) occurs; binding of antigen epitopes to lymphocyte receptors; and production of the appropriate cytokines by antigen presenting cells (APCs), T cells, and B lymphocytes (B cells) that are needed for clonal expansion of antibody-producing and/or cytotoxic cells. There are numerous natural and synthetic molecules that can attract and stimulate these lymphoid cells. In addition, sustained humoral immunity requires persistent exposure of B cells to an antigen, either by retention of non-degraded epitope sequences bound to follicular dendritic cells, or by the continuous release of the antigen from depots or micro particles. This persistence of the antigen is essential for maintaining a high level of antibody production without having to use frequent booster immunizations.

The terminology used to define various methods of immunostimulation is sometimes misleading and often confusing. Historically, the term “adjuvant” has been assigned to any substance which, when administered with an antigen, is able to stimulate a greater immune response to that antigen than if the antigen is administered alone. This increased response is usually affected by nonspecific stimulation of lymphoid cells by the adjuvant. Many adjuvants, such as one unacceptable for human use, Freund's Complete Adjuvant (FCA), can stimulate many of the biological events referred to earlier, whereas others exhibit only some of these activities. Thus, as research to identify better immunopotentiators has progressed, it has become necessary to define the properties of these compounds more precisely, for example, as cellular attractants, macrophage (APC) activators, T-cell mitogens, B-cell mitogens, or antigen delivery systems. Many of these substances have been defined in terms of their principal activity even though they may produce more than one biological effect. Many preparations and immunization procedures exhibit a combination of activities. The present invention focuses on a vaccine formulation method designed to induce elevated antibody levels and to ensure a persistent supply of antigen using a novel delivery system and thus sustain antibody production for a protracted period with minimal side effects.

As examples, several of the newly developed vaccines are designed to treat medical conditions other than infectious diseases. At least two hCG vaccines have been developed for the purpose of contraception or the treatment of cancer. Clinical trials have been conducted with these vaccines and results have been reported. (Talwar G P, Singh O, Pal R, Chatterjee N, Jallai P, Dahil K, Kaus J, Das Sk, Suri S, Buchshee, Saraya T, Saxena B N. A vaccine that prevents pregnancy in women. Proc Natl Acad Sci (USA) 91:8532-6, 1994) and (Moulton H M, Yoshihara, P H, Mason D H, Iversen P L, Trozzi P L. Active Specific Immunotherapy with β-Human Chorionic Gonadotropin Peptide Vaccine in Patients with Metastatic Colorectal Cancer: Antibody Response is Associated with Improved Survival, Clinical Cancer Research 8:2044-2051, 2002). While some of these have shown promise for human use, those demonstrating utility often report a high incidence of pain and/or swelling at the injection site or require multiple immunizations. Such side effects are sometimes intolerable for some patients.

One principal use of the hCG vaccine formulations described herein is for the treatment of cancer. hCG is classified as an oncofetal antigen, an antigen highly expressed during embryogenesis, but in negligible amounts by normal adult tissues. hCG is over-expressed in neoplastic tissues. Although only fragments may be expressed, there is no evidence at present that the hCG expressed by neoplastic cells is mutated. The production of hCG by tumors arising from trophoblastic/germ cells, including choriocarcinoma, hydatidiform mole, and embryonal carcinoma of the testis, is an essential marker of the activity of these diseases, and its measurement plays a central role in their clinical management. That a wide variety of tumors of non-trophoblastic origin also express hCG is also well recognized. Thus the spectrum of expression of hCG by both trophoblastic and non-trophoblastic cancers represents a group of likely targets for vaccine therapy.

Braunstein (Braunstein G D: Placental proteins as tumor markers. In: Immunodiagnosis of Cancer. Herberman R B and Mercer D W (eds) Marcel Dekker, Inc., New York, pp 673-701, 1990) reviewed hCG expression by cancer cells in 1990 and surmised that immunoreactive hCG could be demonstrated in the sera of approximately 18% of patients with non-trophoblastic malignancies. hCG was detected most commonly in the sera of patients with uterine cervical (34%), ovarian (29%), pancreatic (27%), bladder (23%), hepatic (22%), and gastric (21%) cancers. Most of the studies reviewed, however, were based on the recognition of hCG, hCG-like material, or free hCG subunits by either polyclonal or monoclonal antibodies that were not totally specific or of high affinity. Recently, more sensitive, monoclonal-based assays highly specific for intact hCG, α-hCG, β-hCG, the carboxy terminal peptide of the beta subunit (CTP), and the β-core fragment have enabled investigators to re-evaluate the production of hCG by tumors. For example, elevated serum-hCG in 72% of patients with pancreatic cancer and in 9% of those with non-malignant diseases has been found using a highly sensitive time-resolved immunofluorometric assay. Low-levels of hCG and free α-hCG subunit, as measured by an immunoradiometric assay, have been reported to be common in the sera of normal subjects. Free β-hCG (≧100 pg/ml), however, was more tumor specific and was detected in the sera of 47% of bladder, 32% of pancreatic, and 30% of cervical cancer patients, in addition to a majority of patients with germ cell tumors. Normal subjects have free β-hCG serum levels less than 100 pg/ml.

Membrane-associated hCG and/or subunits are a common feature of cultured cancer cell lines. Membrane-associated hCG is also detected frequently on immunohistochemical studies of clinical tumor specimens. In Braunstein's summary, detection of hCG in tumor tissue was most frequent in colorectal (52%), lung (34%), pancreatic (31%), esophageal (28%), breast (24%), and bladder (21%) cancers. Immunohistochemical detection was also reported in approximately 8 to 19% of gastric, prostate, ovarian, uterine cervical, and endometrial cancers. Again, the reported expression of membrane-associated hCG has varied depending on techniques and the patient population studied. The rate of expression in cancers such as gastric and prostate may be higher than initially considered, i.e., greater than 50%. In other studies, the expression of the β-hCG CTP by human colorectal cancer specimens, as determined immunohistochemically with a specific monoclonal antibody, was 85%. Other investigators have reported the detection of hCG on membranes of virtually all cancer cell lines (Acevedo H F, Krichevsky A, Campbell Acevedo E A, Galyon F C, Buffo M J, Hartsock R J. Expression of membrane-associated human chorionic gonadotropin, its subunits and fragments by cultured human cancer cells. Cancer 69:1829-42, 1992).

Use of the whole hCG hormone as a vaccine antigen is not suitable because antibodies raised against it will react with other hormones. Consequently, subunits or peptide fragments specific to hCG have been employed as antigens. Vaccines using such antigens have been tested in humans for both contraception (Talwar G P, Singh O, Pal R, Chatterjee N, Jallai P, Dahil K, A vaccine that prevents pregnancy in women. Proc Natl Acad Sci (USA) 91:8532-6, 1994) and (Jones W R, Judd S J, Ing R M Y, Powell J, Bradley J, Denholm E A, Mueller U W, Griffin P D, Stevens V C. Phase I clinical trial of a World Health Organization birth control vaccine. Lancet. 11: 1295-8, 1988) and for cancer therapy (Moulton H M, Yoshihara, P H, Mason D H, Iversen P L, Trozzi P L. Active Specific Immunotherapy with β-Human Chorionic Gonadotropin Peptide Vaccine in Patients with Metastatic Colorectal Cancer: Antibody Response is Associated with Improved Survival, Clinical Cancer Research 8:2044-2051, 2002) and (Triozii P L, Gochnour D, Martin E W, Aldrich W, Powell J, Kim J A, Young D C, Lombardi J. Clinical and Immunologic effects of a synthetic β-human chorionic gonadotropin vaccine. Int J Oncol 5: 1447-53, 1994). Aluminum salts and water-in-oil emulsions requiring multiple injections have been used in these tests. The vaccines using the alum adjuvant required frequent administrations to maintain an effective antibody level, which made it impractical to use. Likewise, multiple injections of the emulsion-based vaccines, while more effective, sometimes caused an undesirable level of injection site pain and swelling. The widespread application of any of these hCG vaccines is more likely to occur if they are reformulated to use a more suitable adjuvant/delivery system that renders them effective, yet less irritating.

A specific hCG vaccine described herein utilizes conjugates of two peptide segments of (β-hCG conjugated to diphtheria toxoid: the CTP portion (residues 109-145) and a second peptide representing the amino acid 38-57 region. In this embodiment of the invention, an analogue of the 38-57 peptide sequence, rather than the native sequence, is used. The resultant combined immunogen is more potent in inducing hCG reactive antibodies, but more importantly, these antibodies are much more effective in the bio-neutralization of hCG in vitro and in vivo, which can be of value in some applications.

Immunization with vaccines containing the conjugates described above has resulted in increased survival times in patients with metastatic colorectal cancer but did not address the problems of pain and swelling at the injection site. Nor did it address the fact that many vaccine formulations require multiple injections to achieve high and lasting antibody responses (Moulton H M, Yoshihara, P H, Mason D H, Iversen P L, Trozzi P L. Active Specific Immunotherapy with β-Human Chorionic Gonadotropin Peptide Vaccine in Patients with Metastatic Colorectal Cancer: Antibody Response is Associated with Improved Survival, Clinical Cancer Research 8:2044-2051, 2002).

Extensive research has been conducted over the last two decades to identify more suitable systems to deliver vaccines. Many improvements have been made with the use of liposomes, biodegradable polymer microspheres and formulations for mucosal immunizations. Some of these systems are in clinical trials and may prove useful for future vaccines. Another unique system has recently been developed for drug delivery, including vaccine delivery, which offers significant advantages to the other systems under development mentioned above. When used to deliver vaccines, the vaccine antigen is entrapped in an inorganic salt/biopolymer matrix and the solid matrix is ground into very fine particles for use. The micro particles are suspended in an emulsion vehicle for administering to the vaccine recipient. This method, shown to be useful in delivering an hCG vaccine formulation that elicits a highly effective immune response with minimal side effects, comprises this invention.

### SUMMARY OF THE INVENTION

This invention describes a novel method for the formulation of hCG-based vaccines comprising entrapping one or more hCG peptide/diphtheria toxoid (DT) or tetanus toxoid (TT) conjugates into an inorganic/biopolymer matrix and administering them to humans in a water-in-oil based emulsion containing a synthetic adjuvant. The hCG peptides conjugated to the toxoid include those representing beta subunit sequences 109-145 and 38-57 and analogues of the sequences. The aforementioned matrix is comprised of calcium sulfate hemi-hydrate and dextran sulfate. The solid matrix is ground into a fine powder before it is suspended into the emulsion for delivery. Particles of the required size are obtained by sequential sieving through different standard mesh screens. One form of the suspending emulsion may be composed of squalene, mannide monooleate (MM) and phosphate-buffered saline. A synthetic adjuvant compound, such as nor-MDP, may be incorporated into the aqueous portion of the emulsion before emulsification. Doses of the vaccine are administered intramuscularly to recipients. Examples of such formulations and variations thereof have been shown in animal models to elicit high levels of antibodies reactive with hCG for periods exceeding six months from a single inoculation without producing unacceptable local tissue reactions at the injection site. The antibodies produced by these formulations are useful for the treatment of patients with cancer and for preventing pregnancy.

### DETAILED DESCRIPTION OF THE INVENTION

The incorporation of hCG peptide antigens into the inorganic salt/biopolymer matrix is accomplished by methods described by Royer (U.S. Pat. Nos. 6,391,336, 6,497,901 and 6,630,486, by their entirety, incorporated by reference herewith). The inorganic salt employed for use in the current invention is calcium sulfate hemi-hydrate and the biopolymer used is dextran sulfate, the preferred form having an average molecular weight of 8000 daltons. Two of the hCG peptide antigens entrapped represent either hCG beta subunit sequences 109-145 and 38-57 or analogues of these peptides conjugated to DT in a specified ratio of peptide vs. DT or combinations of these conjugates. TT can be substituted for DT. Each conjugate antigen, preferably in a dry state, is mixed separately with calcium sulfate hemi-hydrate in a proportion by weight of 1-10 parts salt to 1 part antigen with the preferred ratio being 4:1. After thorough mixing of the dry components, a volume of aqueous 10 percent dextran sulfate (wt/volume) is added to the appropriate weight of dry conjugate/salt premix with continual stirring to form a slurry with a uniform dispersion of the conjugate within. The weight of the mixture in the wet state is recorded. The mixture is allowed to dry at room temperature, with ventilation, for 2-5 days until no further change in the weight of the mixture is observed. A final weight is recorded before the solid matrix is ground to a fine powder using a mortar and pestle, or mechanical grinder, and the water content of the powder determined. Water content plus loss of water on drying is used to calculate the concentration of antigen in the solid matrix (mass balance). The desired micro-particle sizes for use are obtained by sieving through a series of standard mesh sieves. Particles used for vaccine formulation range from 10-800 micrometers with a preferred size range of 45-150 micrometers.

The dried antigen-containing particles are typically stable to long-term storage at ambient temperature, but may be stored under refrigeration if desired or necessitated by properties of particular antigens.

To formulate a vaccine, the antigen-containing matrix particles are suspended in a liquid medium with sufficient viscosity to maintain a uniform distribution of particles throughout the suspension. Alternately a less viscous liquid medium may be used so long as the particles can be easily resuspended prior to injection. Various liquid vehicles can be used, such as water-in-oil emulsions, oil-in-water emulsions, 0.1-2.0 percent carboxymethyl cellulose, hyaluronic acid solutions, lecithin solutions or other acceptable pharmaceutical vehicles. One preferred vehicle for hCG conjugate antigens is a water-in-oil emulsion of squalene and phosphate-buffered saline (PBS) in a ratio of 3 parts oil phase to 2 parts PBS (v/v). An acceptable oil is provided by a mixture of 4 parts squalene to 1 part MM, which acts as an emulsifier and stabilizes the emulsion. The emulsion is prepared using a mechanical mixer or by hand using hypodermic syringes connected by a three-way stopcock. Other ratios of oil phase vs. PBS are useful for other antigens depending upon their solubility and/or dose required in a vaccine. Other oils, such as squalane, may also be used.

Most weak antigens, including the hCG conjugates used in the current invention, require an adjuvant compound to be administered together with the antigen to stimulate the desired immune response. Such compounds can be added to the matrix and mixed with antigen at the time the matrix is prepared or added to the aqueous phase of the emulsion before the emulsion is formed. Adjuvant compounds that have been shown to be effective for this purpose include mycobacteria, synthetic fragments of bacteria membranes or synthetic analogues of these, aluminum salts, magnesium salts, lipids and numerous other compounds (Adams A. Synthetic Adjuvants. New York: John Wiley & Sons, 1985). A preferred adjuvant for the invention disclosed herein is a synthetic analogue of a bacteria membrane, nor Muramyl Dipeptide (nor MDP), which is chemically defined as N-Acetyl-D-Glucosamine-3-y-L-Ala-D-isoGln Sodium Salt. This compound is highly water-soluble and is added to the aqueous phase of the squalene/PBS emulsion in concentrations such that the final emulsion has a concentration of 5-100 micrograms per milliliter.

For final formulation of a vaccine, a PBS and oil emulsion is first formed in a double syringe system, by forcing the oil/aqueous mixture back and forth between the syringes until a smooth, milky emulsion is achieved. Next, the syringe containing the emulsion is detached and reconnected to a new syringe containing dry antigen particles. The selected dose of matrix particles (determined by antigen concentration in the matrix and the weight of antigen needed) is then suspended in the delivery vehicle by mixing back and forth until an even suspension of particles in the vehicle is achieved. The dry matrix particles may be mixed with the vehicle at the time of immunization or by batch mixing of particles with the vehicle in bulk volumes for dispensing unit doses into sterile vials, ampoules, syringes, or other suitable delivery means. Vaccines formulated in the emulsion preferably are stored at temperatures of 2-8 degrees C.

Antigen concentrations in the matrix can vary from 12-24 percent by weight with a preferred concentration of 16-18 percent. Doses of hCG conjugate for human immunizations can range from 10-500 micrograms and can be administered in 0.1-1.5 milliliters. Selected antigen and adjuvant doses, in the appropriate volume, are injected intramuscularly to recipients at a medically acceptable site. In humans, this normally is in the upper arm, thigh or buttocks.

The release of entrapped antigens is much slower when particles are suspended in the emulsion than when suspended in an aqueous vehicle (FIG. 1).

### DESCRIPTION OF THE FIGURES

FIG. 1 illustrates the comparison of in vitro release rates of the conjugate DT/beta subunit peptide 109-145 (CTP/DT) entrapped in the inorganic/biopolymer matrix, either suspended directly in extracting medium (PBS) or after first emulsifying with squalene/mannide monooleate/PBS emulsion. Particles or emulsified particles were placed in 13×100 mm disposable tubes with aliquots of 1.0 mL PBS, mixed by vortexing and rotated in an incubator at 37 degrees C. At the indicated times, the tubes were centrifuged at low speed (900×g) for 5-7 minutes to settle the particles or partition the emulsion/oil/PBS. The supernatant PBS was drawn off with a micropipet and tested for the presence of extracted conjugate by a Lowry protein assay procedure. Another 1.0 ml of PBS was added back to each tube, and samples were mixed by vortexing and then rotated at 37 degrees C. as before. Curves indicate the total amount of conjugate extracted from particles directly suspended in PBS (diamonds) and particles suspended in emulsion and then mixed with PBS (squares).

FIG. 2 illustrates the mean antibody levels obtained in four rabbits following a single injection of matrix particles containing 1.0 mg of the conjugate DT/hCG beta subunit peptide 109-145 (CTP/DT) in 1.0 ml of a squalene/PBS emulsion containing 0.025 mg of the nor-MDP adjuvant.

FIG. 3 illustrates the mean antibody levels obtained in four rabbits following a single injection of matrix particles containing 1.0 mg of the conjugate DT/beta subunit peptide 38-57 analogue (Loop/DT) in 1.0 ml of a squalene/PBS emulsion containing 0.025 mg of the nor-MDP adjuvant.

FIG. 4 illustrates the mean antibody levels obtained in two groups of rabbits (4-8 rabbits/group) following injections of either (a) three inoculations of vaccine without the conjugate being entrapped in matrix particles (diamonds), or (b) a single inoculation of vaccine with conjugate entrapped in matrix particles (squares), where each formulation contains equal amounts of the conjugates DT/beta subunit peptides 109-145(CTP/DT) and 38-57 analogue (Loop/DT). All doses were given in squalene/PBS emulsion containing 0.025 mg of the nor-MPD adjuvant. Each inoculation involved 0.05 mg of conjugates and was administered to rabbits. For the group receiving matrix-containing vaccine, the single dose of vaccine in 0.5 ml of emulsion was given on day one (left arrow) and no further injections were given. For the group receiving vaccine without matrix particles, doses were given in 1.0 ml of emulsion, on day 1 (left arrow) and again at weeks 3 and 6 (middle and right arrows).

FIG. 5 illustrates the mean antibody levels in three groups of rabbits (4-8 rabbits/group) following injections of matrix particles containing equal amounts of the conjugates DT/beta subunit peptides 109-145(CTP/DT) and 38-57 analogue (Loop/DT) using combined doses of 0.045, 0.090 or 0.135 mg administered to each rabbit on day 1 and at 24 weeks. All doses were given in 0.5 ml of a squalene/PBS emulsion containing 0.025 mg of the nor-MPD adjuvant.

### EXAMPLE I

In order to investigate release of immunogen from the inorganic/biopolymer matrix, an in vitro method was developed to determine the release rate. The conjugate DT/beta subunit peptide 109-145 (CTP/DT) was entrapped in the inorganic/biopolymer matrix, and then either suspended directly in extracting medium (PBS) or suspended in PBS after first suspending the particles in a preformed squalene/MM/PBS emulsion. Particles or emulsified particles were then placed in 13×100 mm disposable tubes with aliquots of 1.0 mL PBS, mixed by vortexing and then rotated in an incubator at 37 degrees C. At specified times, the tubes were centrifuged at low speed (900×g) for 5-7 minutes to settle the particles or partition the emulsion/oil/PBS. The supernatant PBS was drawn off with a micropipet and tested for the presence of extracted conjugate by a Lowry protein assay procedure. Another 1.0 ml of PBS was then added back to each tube, samples were mixed by vortexing and then rotated at 37 degrees C. as before.

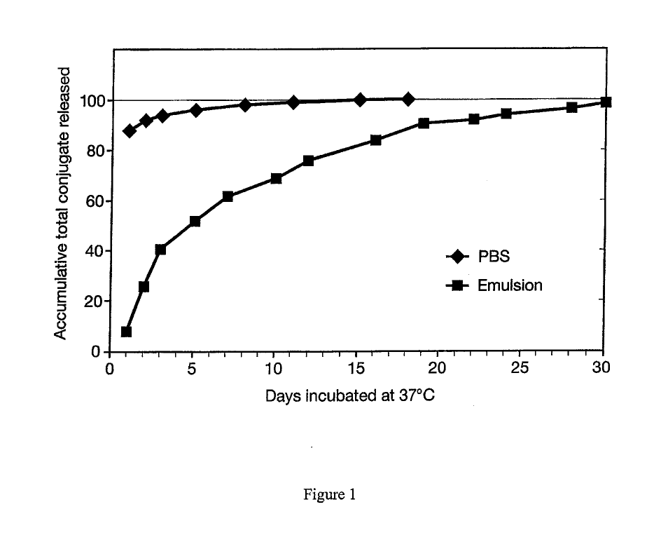

The extraction curves obtained are shown in FIG. 1. In both cases, the inorganic/biopolymer matrix retained significant amounts of the conjugate and released it over a number of days. However, the conjugate extracted much more quickly from particles directly suspended in PBS (diamonds) than from particles suspended in emulsion and then mixed with PBS (squares). These slower release rates of conjugate are likely to be responsible for high titers and long duration of titers seen in immunization studies (Examples II and III, below), because slow release from the injection site is known to augment immune responses to vaccines. Based on the superior retention of conjugate by the emulsified particles, further studies focused on this formulation of the conjugate.

### EXAMPLE II

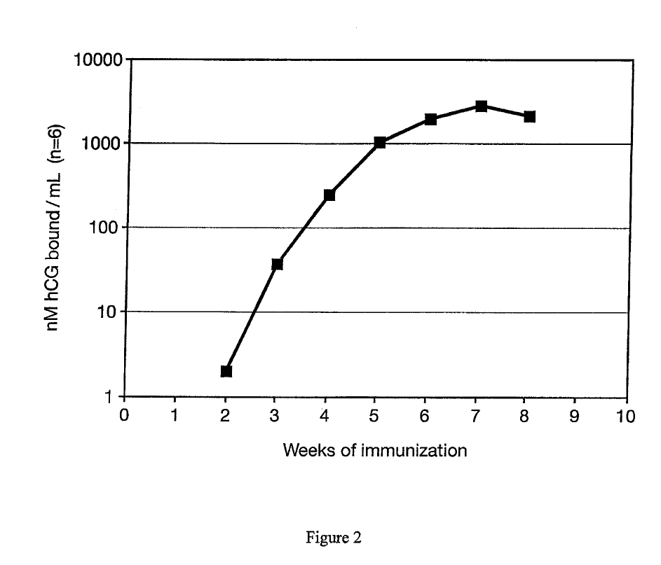

A test of the immunogenicity of the antigen beta hCG peptide 109-145 conjugated to DT (CTP/DT) incorporated in the inorganic/biopolymer matrix was conducted using rabbits as vaccine recipients. Four rabbits were each injected with a formulation containing 1.0 mg of the conjugate in 1.0 ml of an emulsion of squalene/PBS (60%/40% v/v) which contained 0.025 mg of nor MDP. Antibody levels were measured weekly using a radioimmunoassay and expressed as nM/L binding capacity. A single intramuscular injection resulted in the production of elevated antibodies by two weeks after the injection. Peak antibody levels of over 1,500 nM/L were attained by 7 weeks, and were sustained at levels above 100 nM/L for several months. These levels are 40-100 times higher than required in colon cancer patients to provide benefit from therapy (FIG. 2).

This study showed that the formulation was highly effective. Compared to results previously obtained in inoculations with identical amounts of conjugate but where the conjugate was not entrapped in matrix particles, the data indicated that a lower dose of antigen entrapped in the matrix could be used to obtain the necessary level of antibodies for therapy than with the non matrix-entrapped formulation.

### EXAMPLE III

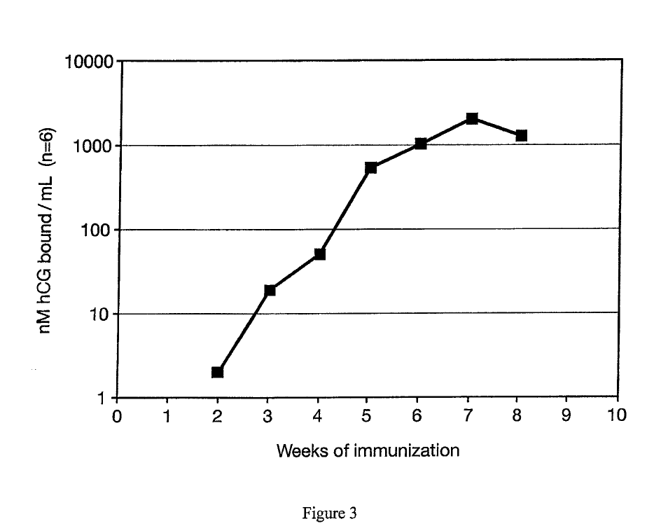

Another test was performed using identical conditions as in Example II except a beta hCG 38-57 analogue peptide conjugated to DT (Loop/DT) was used as the antigen. Very similar results were obtained (FIG. 3). Compared to results previously obtained in inoculations with identical amounts of conjugate but where the conjugate was not entrapped in matrix particles, the data indicated that lower doses of antigen entrapped in the matrix were more effective for use in a vaccine than the non matrix-entrapped formulation.

### EXAMPLE IV

A comparison of the efficacy of vaccines identical except for the presence or absence of the inorganic/biopolymer matrix was conducted using rabbits as vaccine recipients.

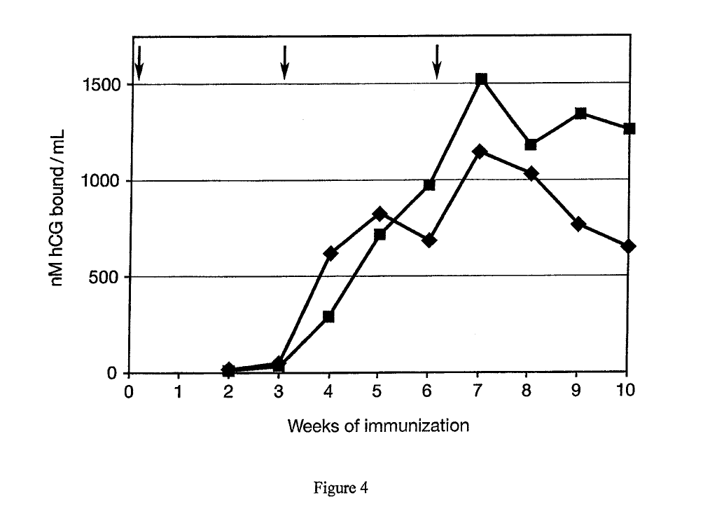

Four rabbits were each injected intramuscularly with a formulation containing 0.5 mg of each of the two conjugates (CTP/DT and Loop/DT as in examples II and III above) in 0.5 ml of an emulsion of squalene/PBS (60%/40%) which contained 0.025 mg of nor MDP. Antibody levels were measured weekly using a radioimmunoassay and expressed as nM/L binding capacity. A single intramuscular injection of inorganic/biopolymer-matrix-containing vaccine was compared to three injections of vaccine lacking the inorganic/biopolymer matrix, in order to achieve approximate parity of effects. FIG. 4 shows that both vaccination regimens resulted in the production of elevated antibodies by two weeks after the injection. However, titers obtained from the injections of inorganic/biopolymer-matrix-containing vaccine reached significantly higher levels and remained higher than those from vaccine lacking the inorganic/biopolymer matrix, despite the latter having been injected three times vs. one time for the inorganic/biopolymer-matrix-containing vaccine.

Table 1 compares the tissue reactions obtained for the immunizations. For the vaccine not entrapped in the inorganic/biopolymer matrix, the tissue reactions at the injection sites, measured as in Example VI, were minimal to moderate in all animals, but the average reaction (lesion score) was 1.4, substantially above the clinically acceptable level of 1.0. In contrast, the tissue reactions were negligible for the inorganic/biopolymer-matrix-containing vaccine, and well within clinically acceptable limits.

TABLE 1
Tissue reactions at injection sites.

| | |
|---|---|
| Without matrix | With matrix |

| Rabbit | A | B | C | D | E | F | G | H |
|---|---|---|---|---|---|---|---|---|
| Lesion | 0.5 | 1.0 | 2.0 | 2.0 | 0.0 | 0.0 | 0.0 | 0.0 |
| score* | | | | | | | | |

| | | |
|---|---|---|
| Group | 1.4 | 0.0 |
| average | | |

*Measured as described in Example VI

### EXAMPLE V

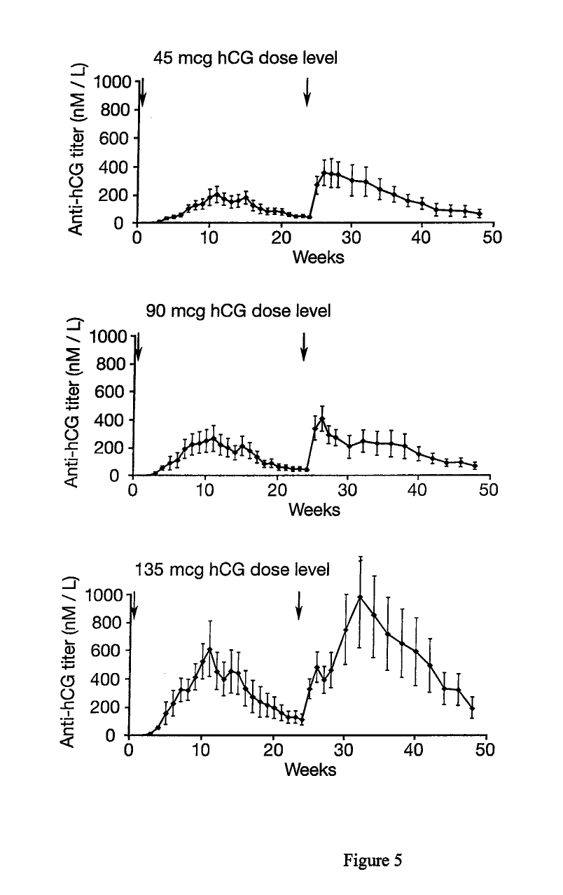

A series of tests using individual conjugates indicated that safe and effective doses of each hCG conjugate incorporated into the matrix was between 0.010 and 0.10 mg. A study was then initiated in which a dose response was obtained for a combination of both CTP/DT and Loop/DT antigens in equal proportions. Doses containing a total of 0.045-0.135 mg of the combined conjugates (50/50 ratio, w/w) were tested for their ability to induce hCG-specific antibody production in rabbits. Injections were given on day 1 and at 24 weeks from the first injection. The antibody profiles from this study are shown in FIG. 5. Antibody levels, even at the lowest dose studied, were more than adequate for clinical use.

The tissue reactions measured as in Example VI were minimal at the lowest doses and moderate, but acceptable, at the highest doses used.

### EXAMPLE VI

### Macroscopic Evaluation of Tissue Reactions at the Injection Site

In order to evaluate the gross (macroscopic) appearance of the thigh muscle after injection of test materials, the skin of euthanized animals was peeled off the examined thigh by making a transverse and longitudinal incision and then peeling off the skin. Care is taken to make a clean separation from muscle tissue, without damaging the latter. Using a sharp lancet, each injection site was incised to expose the interior of the thigh muscle. Additional incisions were made if necessary to ensure complete viewing and assessment of pathology. Biopsy specimens were preserved in 10% buffered formalin.

### Scale for Evaluation of Gross Appearance:

### 0: Normal Tissue

No visible pathology. At times yellow fatty/fibrous tissue appeared after complete resolution of inflammation in the muscle tissue. Such a change was not rated as pathological.

### 1: Minimal Pathology

A typical appearance included small (<3 mm diameter) hard nodules representing encapsulated and resolving sterile abscesses or inflammatory sites. The combined volume of such lesions was required to be less than 5% of the total thigh muscle volume.

### 2: Moderate Pathology

Nodules were larger (3-10 mm diameter) and included hard to the touch (old fibrosis) or soft (more recently encapsulated) lesions. On squeezing such lesions, pus or injection material sometimes was expressed. Free (unencapsulated) material was occasionally seen. In that case, the longitudinal diameter of the lesion was required to be no larger than 10 mm. The combined volume of the lesions was required to be between 5-10% of the total thigh muscle volume.

### 3: Several Pathology

Large, encapsulated or unencapsulated lesions, greater than 10 mm in longitudinal diameter. Typically, lesions contained pus (sterile abscesses) or injection material (emulsion). Total volume of the lesions was >10% of total thigh muscle volume.

### Intermediate Grades:

When lesions did not fall unequivocally within the definition of a certain grade, intermediate grades were designated as 0-1, 1-2, or 2-3. To calculate group means, intermediate grades were assigned half values (for example, 0-1 was assigned a value of 0.5).

What is claimed is:

1. A method for delivering an antigen effective for eliciting an antibody against Human Chorionic Gonadotropin (hCG) comprising:
incorporating the antigen into an inorganic/biopolymer matrix,
suspending the antigen-incorporated matrix in a water-in-oil emulsion containing an adjuvant, and
injecting into humans the emulsion containing the antigen-incorporated matrix for treating patients suffering from cancer.

2. The method according to claim 1, wherein the antigen is a conjugate of a carrier protein such as diphtheria toxoid and a peptide of the hCG beta subunit sequence of 109-145 or an analogue peptide of the hCG beta subunit sequence of 109-145.

3. The method according to claim 1, wherein the antigen is a conjugate of a carrier protein and a peptide of the hCG beta unit sequence of 38-57 or an analogue peptide of the hCG beta unit sequence of 38-57.

4. The method according to claim 1, wherein the antigen is any combination of
a conjugate of a carrier protein and a peptide of the hCG beta subunit sequence of 109-145,
a conjugate of a carrier protein and an analogue peptide of the hCG beta subunit sequence of 109-145,
a conjugate of a carrier protein and a peptide of the hCG beta unit sequence of 38-57,
a conjugate of a carrier protein and an analogue peptide of the hCG beta unit sequence of 38-57, or
a conjugate of a carrier protein and a peptide of the hCG beta subunit.

5. The method according to claim 1, wherein the inorganic/biopolymer matrix comprises a calcium sulfate hemi-hydrate/dextran sulfate matrix, or a calcium phosphate/dextran sulfate matrix.

6. The method according to claim 1, wherein the water-in-oil emulsion comprises squalene and phosphate-buffered saline (PBS).

7. The method according to claim 6, wherein the emulsion further comprises an emulsifier.

8. The method according to claim 7, wherein the emulsifier is mannide monooleate.

9. The method according to claim 8, wherein the squalene and mannide monooleate are mixed at a ratio of 4:1.

10. The method according to claim 9, wherein the squalene: mannide monooleate mixture serves as an oil phase of the emulsion.

11. The method according to claim 10, wherein the oil phase of the emulsion is 60 percent by volume of the emulsion and PBS is 40 percent.

12. The method according to claim 1, wherein the adjuvant is N-Acetyl-D-Glucosamine-3-y-L-Ala-D-isoGln Sodium Salt (nor MDP).

13. The method according to claim 1, whereby a vaccine to treat patients suffering from colon cancer is formulated.

14. The method according to claim 1, whereby vaccine to treat patients suffering from pancreatic cancer is formulated.

15. The method according to claim 1, whereby a vaccine to treat patients suffering from bladder cancer is formulated.

16. The method according to claim 1, whereby a vaccine to treat patients suffering from prostate cancer is formulated.

17. The method according to claim 1, whereby a vaccine to treat patients suffering from ovarian cancer is formulated.

18. The method according to claim 1, whereby a vaccine to treat patients suffering from lung cancer is formulated.

19. The method according to claim 1, whereby a vaccine to treat patients suffering from cervical cancer is formulated.

20. The method according to claim 1, whereby a vaccine to treat patients suffering from breast cancer is formulated.

21. The method according to claim 1, whereby a vaccine to treat patients suffering from renal cancer is formulated.

22. The method according to claim 1, whereby a vaccine to treat patients suffering from hepatic cancer is formulated.

23. The method according to claim 1, whereby a vaccine to treat patients suffering from melanoma cancer is formulated.

24. A method for delivering an antigen effective for eliciting an antibody against Human Chorionic Gonadotropin (hCG) comprising:
incorporating the antigen into an inorganic/biopolymer matrix,
suspending the antigen-incorporated matrix in a water-in-oil emulsion containing an adjuvant, and
injecting into humans the emulsion containing the antigen-incorporated matrix for treating patients suffering from a hormone-related disease or preventing a hormone-related condition, wherein the hormone-related disease is selected from a group consisting of hydatidiform mole, ectopic pregnancy, and cancer.

25. The method according to claim 24, wherein the antigen is a conjugate of a carrier protein such as diphtheria toxoid and a peptide of the hCG beta subunit sequence of 109-145 or an analogue peptide of the hCG beta subunit sequence of 109-145.

26. The method according to claim 24, wherein the antigen is a conjugate of a carrier protein such as diphtheria toxoid and a peptide of the hCG beta unit sequence of 38-57 or an analogue peptide of the hCG beta unit sequence of 38-57.

27. The method according to claim 24, wherein the antigen is any combination of
a conjugate of a carrier protein and a peptide of the hCG beta subunit sequence of 109-145,
a conjugate of a carrier protein and an analogue peptide of the hCG beta subunit sequence of 109-145,
a conjugate of a carrier protein and a peptide of the hCG beta unit sequence of 38-57,
a conjugate of a carrier protein and an analogue peptide of the hCG beta unit sequence of 38-57, or
a conjugate of a carrier protein and a peptide of the hCG beta subunit.

28. The method according to claim 24 where the hormone-related condition being prevented is pregnancy.

29. The method according to claim 24, wherein the inorganic/biopolymer matrix comprises a calcium sulfate hemi-hydrate/dextran sulfate matrix, or a calcium phosphate/dextran sulfate matrix.

30. The method according to claim 24, wherein the water-in-oil emulsion comprises squalene and phosphate-buffered saline (PBS).

31. The method according to claim 24, wherein the emulsion further comprises an emulsifier.

32. The method according to claim 31, wherein the emulsifier is mannide monooleate.

33. The method according to claim 32, wherein the squalene and mannide monooleate are mixed at a ratio of 4:1.

34. The method according to claim 33, wherein the squalene: mannide monooleate mixture serves as an oil phase of the emulsion.

35. The method according to claim 34, wherein the oil phase of the emulsion is 60 percent by volume of the emulsion and PBS is 40 percent.

36. The method according to claim 24, wherein the adjuvant is N-Acetyl-D-Glucosamine-3-y-L-Ala-D-isoGln Sodium Salt (nor MDP).

37. A method for delivering an antigen effective for eliciting an antibody against Human Chorionic Gonadotropin (hCG) comprising:
incorporating the antigen into an inorganic salt/biopolymer matrix,
suspending the antigen-incorporated matrix in a water-in-oil emulsion containing an adjuvant, and
injecting into humans the emulsion containing the antigen-incorporated matrix for treating patients suffering from an hCG-related disease or preventing an hCG-related condition.

38. The method according to claim 37, wherein the inorganic salt/biopolymer matrix comprises calcium sulfate hemi-hydrate/dextran sulfate matrix.

39. The method according to claim 37, wherein the inorganic salt is selected from a group consisting of calcium sulfate hemi-hydrate, calcium sulfate, and calcium phosphate, and the biopolymer is selected from a group consisting of hyaluronic acid, chondroitin sulfate, protein, glycosaminoglycan, dextran, dextran sulfate, starch, xanthan, chitosan, cellulose and cellulose derivatives.

40. The method according to claim 37, wherein the antigen is a conjugate of a carrier protein such as diphtheria toxoid and a peptide of the hCG beta subunit sequence of 109-145 or an analogue peptide of the hCG beta subunit sequence of 109-145.

41. The method according to claim 37, wherein the antigen is a conjugate of a carrier protein such as diphtheria toxoid and a peptide of the hCG beta unit sequence of 38-57 or an analogue peptide of the hCG beta unit sequence of 38-57.

42. The method according to claim 37, wherein the antigen is any combination of
a conjugate of a carrier protein and a peptide of the hCG beta subunit sequence of 109-145,
a conjugate of a carrier protein and an analogue peptide of the hCG beta subunit sequence of 109-145,
a conjugate of a carrier protein and a peptide of the hCG beta unit sequence of 38-57,
a conjugate of a carrier protein and an analogue peptide of the hCG beta unit sequence of 38-57, or
a conjugate of a carrier protein and a peptide of the hCG beta subunit.

43. The method according to claim 37 where the hormone-related condition being prevented is pregnancy.

44. The method according to claim 37, wherein the inorganic/biopolymer matrix is a calcium sulfate hemi-hydrate/dextran sulfate matrix, or a calcium phosphate/dextran sulfate matrix.

45. The method according to claim 37, wherein the water-in-oil emulsion comprises squalene and phosphate-buffered saline (PBS).

46. The method according to claim 37, wherein the emulsion further comprises an emulsifier.

47. The method according to claim 46, wherein the emulsifier is mannide monooleate.

48. The method according to claim 47, wherein the squalene and mannide monooleate are mixed at a ratio of 4:1.

49. The method according to claim 48, wherein the squalene: mannide monooleate mixture serves as an oil phase of the emulsion.

50. The method according to claim 49, wherein the oil phase of the emulsion is 60 percent by volume of the emulsion and PBS is 40 percent.

51. The method according to claim 37, wherein the adjuvant is N-Acetyl-D-Glucosamine-3-y-L-Ala-D-isoGln Sodium Salt (nor MDP).

52. The method according to claim 37, whereby a vaccine to treat patients suffering from colon cancer is formulated.

53. The method according to claim 37, whereby a vaccine to treat patients suffering from pancreatic cancer is formulated.

54. The method according to claim 37, whereby a vaccine to treat patients suffering from bladder cancer is formulated.

55. The method according to claim 37, whereby a vaccine to treat patients suffering from prostate cancer is formulated.

56. The method according to claim 37, whereby a vaccine to treat patients suffering from ovarian cancer is formulated.

57. The method according to claim 37, whereby a vaccine to treat patients suffering from lung cancer is formulated.

58. The method according to claim 37, whereby a vaccine to treat patients suffering from cervical cancer is formulated.

59. The method according to claim 37, whereby a vaccine to treat patients suffering from breast cancer is formulated.

60. The method according to claim 37, whereby a vaccine to treat patients suffering from renal cancer is formulated.

61. The method according to claim 37, whereby a vaccine to treat patients suffering from hepatic cancer is formulated.

62. The method according to claim 37, whereby a vaccine to treat patients suffering from melanoma cancer is formulated.

63. The method according to claim 37, wherein the hCG-related disease is selected from a group consisting of hydatidiform mole, ectopic pregnancy and cancer.

64. A method for delivering an antigen effective for eliciting an antibody against Human Chorionic Gonadotropin (hCG) comprising:
conjugating a peptide antigen to a carrier to form the conjugated antigen,
incorporating the conjugated antigen into an inorganic/biopolymer matrix, wherein the conjugated antigen is mixed with calcium sulfate hemi-hydrate and dextran sulfate thereby entrapped in a calcium sulfate hemi-hydrate/dextran sulfate matrix,
drying the matrix entrapping the conjugated antigen,
suspending the dried matrix in a water-in-oil or oil-in water emulsion containing an adjuvant, and
injecting into humans the emulsion containing the matrix entrapping the conjugated antigen for treating patients suffering from an hCG-related disease or prevent an hCG-related condition.

65. The method of claim 64, wherein the hCG-related disease is selected from a group consisting hydatidiform mole, ectopic pregnancy, and cancer.

66. The method according to claim 64, wherein the antigen is a conjugate of a carrier protein such as diphtheria toxoid and a peptide of the hCG beta subunit sequence of 109-145 or an analogue peptide of the hCG beta subunit sequence of 109-145.

67. The method according to claim 64, wherein the antigen is a conjugate of a carrier protein such as diphtheria toxoid and a peptide of the hCG beta unit sequence of 38-57 or an analogue peptide of the hCG beta unit sequence of 38-57.

68. The method according to claim 64, wherein the antigen is any combination of
a conjugate of a carrier protein and a peptide of the hCG beta subunit sequence of 109-145,
a conjugate of a carrier protein and an analogue peptide of the hCG beta subunit sequence of 109-145,
a conjugate of a carrier protein and a peptide of the hCG beta unit sequence of 38-57,
a conjugate of a carrier protein and an analogue peptide of the hCG beta unit sequence of 38-57, or
a conjugate of a carrier protein and a peptide of the hCG beta subunit.

69. The method according to claim 64 where the hormone-related condition being prevented is pregnancy.

70. The method according to claim 64, wherein the inorganic/biopolymer matrix comprises a calcium sulfate hemi-hydrate/dextran sulfate matrix, or a calcium phosphate/dextran sulfate matrix.

71. The method according to claim 64, wherein the water-in-oil emulsion comprises squalene and phosphate-buffered saline (PBS).

72. The method according to claim 71, wherein the emulsion further comprises an emulsifier.

73. The method according to claim 72, wherein the emulsifier is mannide monooleate.

74. The method according to claim 73, wherein the squalene and mannide monooleate are mixed at ratio of 4:1.

75. The method according to claim 74, wherein the squalene: mannide monooleate mixture serves as an oil phase of the emulsion.

76. The method according to claim 75, wherein the oil phase of the emulsion is 60 percent by volume of the emulsion and PBS is 40 percent.

77. The method according to claim 64, wherein the adjuvant is N-Acetyl-D-Glucosamine-3-y-L-Ala-D-isoGln Sodium Salt (nor MDP).

78. The method according to claim 64, whereby a vaccine to treat patients suffering from colon cancer is formulated.

79. The method according to claim 64, whereby a vaccine to treat patients suffering from pancreatic cancer is formulated.

80. The method according to claim 64, whereby a vaccine to treat patients suffering from bladder cancer is formulated.

81. The method according to claim 64, whereby a vaccine to treat patients suffering from prostate cancer is formulated.

82. The method according to claim 64, whereby a vaccine to treat patients suffering from ovarian cancer is formulated.

83. The method according to claim 64, whereby a vaccine to treat patients suffering from lung cancer is formulated.

84. The method according to claim 64, whereby a vaccine to treat patients suffering from cervical cancer is formulated.

85. The method according to claim 64, whereby a vaccine to treat patients suffering from breast cancer is formulated.

86. The method according to claim 64, whereby a vaccine to treat patients suffering from renal cancer is formulated.

87. The method according to claim 64, whereby a vaccine to treat patients suffering from hepatic cancer is formulated.

88. The method according to claim 64, whereby a vaccine to treat patients suffering from melanoma cancer is formulated.

89. A vaccine-inorganic/biopolymer matrix composition comprising:
(a) a fragment of hCG beta subunit,
(b) a calcium salt, and
(c) a matrix polymer,
wherein the fragment is substantially entrapped in the inorganic/biopolymer matrix formed by the calcium salt and the matrix polymer.

90. The vaccine-inorganic/biopolymer matrix composition according to claim 89, wherein the antigen is any combination of a conjugate of a carrier protein and a peptide of the hCG beta subunit sequence of 109-145,
a conjugate of a carrier protein and an analogue peptide of the hCG beta subunit sequence of 109-145,
a conjugate of a carrier protein and a peptide of the hCG beta unit sequence of 38-57,
a conjugate of a carrier protein and an analogue peptide of the hCG beta unit sequence of 38-57, or
a conjugate of a carrier protein and a peptide of the hCG beta subunit.

91. The vaccine-inorganic/biopolymer matrix composition according to claim 89, wherein the calcium salt is selected from the group consisting of calcium sulfate hemihydrate, calcium silicates, aluminates, hydroxides, phosphates, and calcium phosphate.

92. The vaccine-inorganic/biopolymer matrix composition according to claim 89, wherein the matrix polymer is selected from the group consisting of dextran, dextran sulfate, hyaluronic acid, chondroitin sulfate, protein, glycosaminoglycan, chitosan, starch, xanthan, cellulose, cellulose derivatives, collagen, fibrinogen, polyglutamic acid, polyaspartic acid, polynucleotides, cationic polypeptides, and defatted albumin.

93. The vaccine-inorganic/biopolymer matrix composition according to claim 89, wherein the calcium salt is calcium sulfate hemihydrate.

94. The vaccine-inorganic/biopolymer matrix composition according to claim 89, wherein the calcium salt is calcium sulfate.

95. The vaccine-inorganic/biopolymer matrix composition according to claim 89, wherein the calcium salt is calcium phosphate.

96. The vaccine-inorganic/biopolymer matrix composition according to claim 89, wherein the matrix polymer is dextran.

97. The vaccine-inorganic/biopolymer matrix composition according to claim 89, wherein the matrix polymer is dextran sulfate.

98. The vaccine-inorganic/biopolymer matrix composition according to claim 89, wherein the inorganic/biopolymer matrix is a calcium sulfate hemi-hydrate/dextran sulfate matrix, or a calcium phosphate/dextran sulfate matrix.
